(19) United States
(12) Patent Application Publication
(10) Pub. No.: US 2026/0242562 A1
(43) Pub. Date: Aug. 20, 2026

(54) INOCULUM COMPOSITION FOR ACCELERATING ANAEROBIC BIODEGRADATION OF PLASTICS AND METHODS FOR PRODUCING THE SAME

(71) Applicant: Nano and Advanced Materials Institute Limited, Hong Kong (CN)

(72) Inventors: Kwan-Wing Cheu, Hong Kong (CN); Xu Zhou, Hong Kong (CN); Sik-Chun Johnny Lo, Hong Kong (CN); Sui-Han Sin, Hong Kong (CN); Yeuk-Tin Lau, Hong Kong (CN)

(21) Appl. No.: 19/058,591

(22) Filed: Feb. 20, 2025

(51) Int. Cl. C08J 11/10; C12N 9/18; C12N 9/20; C12N 9/50

(52) U.S. Cl. CPC C08J 11/105; C12N 9/18; C12N 9/20; C12N 9/50; C12Y 304/21062; C12Y 304/21064; C12Y 304/24039; C08J 2323/06; C08J 2323/12; C08J 2367/04

(57) ABSTRACT

The present invention discloses an inoculum composition, comprising anaerobic condition-surviving bacteria for accelerating anaerobic biodegradation of plastics and/or enzyme, wherein the anaerobic condition-surviving bacteria comprises one selected from the group consisting of Bacillus infernus, Defluviitoga tunisiensis, Syntrophaceticus schinkii, Tepidanaerobacter syntrophicus Tepidimicrobium xylanilyticum and any combination thereof.

### TECHNICAL FIELD

The present invention relates to the technical field about anaerobic biodegradation, in particular to accelerating anaerobic biodegradation of plastics.

### BACKGROUND

Synthetic plastic products derived from petroleum, such as polyethylene terephthalate, polystyrene, and polyethylene, are widely used, especially in the form of food packaging, beverage bottles, and food trays, owing to their low cost and efficient function in preserving and protecting food products.

Growing concern regarding conventional plastics and huge environmental problem caused by these materials has promoted interest in biodegradable plastics. Biodegradable plastics offer additional waste management options (e.g., anaerobic digestion, anaerobic biodegradation) over conventional plastics. However, the treatment of biodegradable plastics under anaerobic digestion or anaerobic biodegradation is only in its infancy with notably no specification standard considering the end-of-life of biodegradable plastics in anaerobic digestion system as a stand-alone.

### SUMMARY

The present invention provides a bioactive inoculum composition for the accelerated anaerobic biodegradation test of the typical plastic polyethylene (PE), polypropylene (PP) and polylactic acid (PLA), respectively, by means of analyzing a series of bioactivity and constructing optimal microbial consortium through considering the compatibility with the soil being bioaugmented and optimization of anaerobic biodegradation rate.

In one embodiment, the present invention discloses an inoculum composition, comprising anaerobic condition-surviving bacteria for accelerating anaerobic biodegradation of plastics and/or enzyme, wherein the anaerobic condition-surviving bacteria comprises one selected from the group consisting of Bacillus infernus, Defluviitoga tunisiensis, Syntrophaceticus schinkii, Tepidanaerobacter syntrophicus Tepidimicrobium xylanilyticum and any combination thereof.

In another embodiment, the present invention provides an inoculum composition with more additives, also referred to “inoculum matrix”, similar to the inoculum composition in the above-mentioned embodiment. The “inoculum matrix” further comprises:
(a) enzyme, for enhancing the biodegradation of plastics; and
(b) conditioning agent, for enhancing the performance of enzyme.

In still another embodiment, the present invention discloses a method for anaerobic biodegradation of plastics, comprising adding the inoculum composition or the inoculum matrix to a system comprising plastics solely or together with anaerobic microbial habitat as bioaugmentation.

In further another embodiment, the present invention provides a method for increasing the proliferative rate of anaerobic condition-surviving bacteria which have been subjected to cryopreservation with cryoprotectant to form a freezing solution. The method comprises:
(a) adding a first amount of the freezing solution into a second amount of recovery solution to form a mixture solution, so as to thaw the anaerobic condition-surviving bacteria, wherein the second amount is 10 times or more than the first amount; and
(b) culturing the thawed anaerobic condition-surviving bacteria in the mixture solution;
wherein the mixture solution is substantially free of gelling agent.

The above description is only an outline of the technical schemes of the present invention. Preferred embodiments of the present invention are provided below in conjunction with the attached drawings to enable one with ordinary skill in the art to better understand said and other objectives, features and advantages of the present invention and to make the present invention accordingly.

### BRIEF DESCRIPTION OF THE DRAWINGS

The present invention can be more fully understood by reading the following detailed description of the preferred embodiments, with reference made to the accompanying drawings, wherein:

FIG. 1 illustrates the stages of anaerobic biodegradation of polymer.

FIG. 2 illustrates the workflow of 16S rRNA and ITS amplicon sequencing.

FIG. 3 exhibits the relative abundance of bacterial community of Soil 1 at the species levels before (Day 0) and after 45 days of PP/PE/PLA-enrichment.

FIG. 4 exhibits the relative abundance of bacterial community of Soil 2 at the species levels before (Day 0) and after 45 days of PP/PE/PLA-enrichment.

FIG. 5 exhibits the relative abundance of bacterial community of Soil 4 at the species levels before (Day 0) and after 45 days of PP/PE/PLA-enrichment.

FIG. 6 illustrates OD600 of four anaerobic bacteria using traditional and accelerated cultivation method.

Experimental setup for anaerobic biodegradation test FIG. 7A shows reference from ASTM D5511; and FIG. 7B shows experiment setup used in NAMI's laboratory.

FIG. 8 illustrates the equation for calculating the percentage of biodegradation.

FIG. 9 shows 90-day percentage acceleration of anaerobic biodegradation of PE in presence of inoculum composition.

FIG. 10 shows 90-day percentage acceleration of anaerobic biodegradation of PP in presence of inoculum composition.

FIG. 11 shows 90-day percentage acceleration of anaerobic biodegradation of PLA in presence of inoculum composition.

FIG. 12 exhibits 90-day percentage acceleration of anaerobic biodegradation of PLA in presence of Lipase.

FIG. 13 exhibits 90-day percentage acceleration of anaerobic biodegradation of PLA in presence of Esterase.

FIG. 14 exhibits 90-day percentage acceleration of anaerobic biodegradation of PLA in presence of Alcalase.

FIG. 15 exhibits 90-day percentage acceleration of anaerobic biodegradation of PLA in presence of Proteinase K and neutral protease BR.

FIG. 16 shows 90-day percentage acceleration of anaerobic biodegradation of PLA in presence of inoculum composition and inoculum matrix respectively.

FIG. 17 shows 90-day anaerobic biodegradation of PLA in presence of inoculum compositions and different enzyme activity of lipase.

### DETAILED DESCRIPTION

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

### Definitions

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The relative abundance of the number of individual physical microbes in the set with respect to the total number of individual physical microbes in the microbiome composition can be determined by quantitating operational taxonomic units (OTUs) or metagenome-assembled genomes (MAGs), as described in the following examples.

It should be understood that the order of steps or order for performing certain actions is immaterial so long as the present teachings remain operable. Moreover, two or more steps or actions may be conducted simultaneously.

Unless defined otherwise, all technical and scientific terms used herein have the same meaning as commonly understood to one of ordinary skill in the art to which the presently described subject matter pertains.

### Anaerobic Digestion (AD)

Anaerobic digestion (AD) is a waste management method allowing a simultaneous generation of renewable energy (i.e., through biogas generation) and treatment of organic wastes. This process uses microorganisms in an oxygen-free environment. The main products are biogas (mainly composed of methane and carbon dioxide) and digestate (the non-degraded fraction, which is rich in mineralized nutrients). Biogas can be valorized by different methods: converted in heat and electricity, upgraded into biomethane for introduction in the gas grid or used as biofuel. Digestate is mostly used as fertilizers or soil amenders.

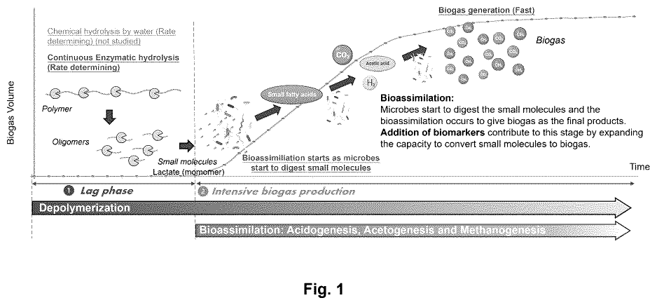

FIG. 1 exhibits schematic illustration of anaerobic biodegradation of polymer and involves four stages: hydrolysis, acidogenesis, acetogenesis, and methanogenesis. First, at the lag phase, microbes or enzymes approach the surface of the polymer and cut the polymer chains into oligomers through random chain scission (e.g. hydrolysis and biological scission). The amorphous structure is susceptible to degradation. At this stage, biogas is usually not detected. Later, the oligomers will further be digested by microbes to produce biogas.

The overall biodegradation rate can be enhanced by shortening the lag phase. If polymer chains are effectively scissored, more oligomers can be obtained to produce more biogas for enhancing the biodegradation.

### Temperature of Anaerobic Digestion (AD)

One of the main factors that affects the efficiency of AD is temperature. Most industrial application are conducted as mesophilic (30-40 degrees Celsius) anaerobic digestion (MAD), and thermophilic (50-60 degrees Celsius) anaerobic digestion (TAD) attracts growing attentions. TAD has been found to have higher biomethane production, a faster reaction rate, higher organic reduction, and a shorter retention time compared to MAD. However, TAD also requires more energy to heat the digester and can be more unstable due to the accumulation of volatile fatty acids (VFA) and inhibition by free ammonia.

### Decomposition of Polylactic Acid (PLA)

Polylactic acid (PLA) is one of the most promising biomass-based and biodegradable plastics, showing a wide range of potential applications. The development of the ring-opening polymerization of lactide has increased the production capacity of high-molecular weight (MW) PLA.

The decomposition of PLA is generally thought to occur through a two-stage mechanism beginning with abiotic PLA hydrolysis, and leading to biotic breakdown by microorganisms.

Some studies proposed an explanation for the low biodegradation rate of PLA. According to their theory, bacteria would not be able to handle high molecular weight PLA. They would be able to use this PLA only after a reduction of its molar mass caused by hydrolysis. Hydrolysis rate of PLA is relatively low at mesophilic conditions; by contrast, at thermophilic conditions the hydrolysis rate is increased.

In a first embodiment, a method to produce functional microbial consortium is provided. The method comprises:
culturing microbes of samples 1 to N obtained from sites 1 to N, wherein the samples 1 to N contacts with polyethylene (PE), polypropylene (PP) or polylactic acid (PLA) under anaerobic condition, N is a nature number equal to or larger than 2;
analyzing and calculating the relative abundance of microbes in each sample at a first time interval under anaerobic biodegradation of polyethylene (PE), polypropylene (PP) or polylactic acid (PLA), more preferred, the first time interval equal to or larger than 40 days; and
selecting microbes that exhibited increased relative abundance of polyethylene (PE), polypropylene (PP) or polylactic acid (PLA) enrichment in at least two samples to produce the microbial consortium.

In this embodiment, the sample is selected from the group consisting of biomass, kitchen waste, volatile fatty acids, sewerage, municipal waste, refinery wastewater, petrochemical industry wastewater, sugar industry waste, slaughter house waste, paper and pulp industry waste, refinery effluent treatment plant (ETP) biosludge, agricultural residues, food waste, domestic waste in landfill or a combination thereof.

Preferred, the microbial consortium is effective at both mesophilic and thermophilic anaerobic conditions. The range of operating temperature is 20 to 60 degrees Celsius.

In one case, the method further comprises selecting microbes that exist in at least one sample at a second time interval under anaerobic biodegradation of polyethylene (PE), polypropylene (PP) or polylactic acid (PLA), and the second time interval is longer than the first time interval. Preferred, the second time interval is equal to or larger than 90 days.

In another case, the method further comprises selecting microbes that exist under anaerobic biodegradation of polyethylene (PE), polypropylene (PP) or polylactic acid (PLA) at a third time interval, longer than the first time interval, which is reported in literature. Preferred, the third time interval is equal to or larger than 90 days.

In a second embodiment, we provide a method for increasing the proliferative rate of anaerobic condition-surviving bacteria which have been subjected to cryopreservation with cryoprotectant to form a freezing solution. The method comprises:
(a) adding a first amount of the freezing solution into a second amount of recovery solution to form a mixture solution, so as to thaw the anaerobic condition-surviving bacteria, wherein the second amount is 10 times or more than the first amount; and
(b) culturing the thawed anaerobic condition-surviving bacteria in the mixture solution;
wherein the mixture solution is substantially free of hydrogel.

Preferred, the method is a one-pot process to cultivate the anaerobic condition-surviving bacteria. By using large amount of recovery solution to directly thaw and culture the anaerobic condition-surviving bacteria, rather than stepwise, slightly increasing the recovery solution together with the anaerobic condition-surviving bacteria on hydrogel plate, such as agar plate, the cultivation time is dramatically decreased 50% or more. In one case, the anaerobic condition-surviving bacteria can be amplified within 1 week from directly sub-culturing the thawed cryopreserved culture.

As mentioned above, the second amount of recovery solution is 10 times or more than the first amount of the freezing solution; preferred 15 times or more; and further preferred 20 times or more.

In this embodiment, the anaerobic condition-surviving bacteria comprises one selected from the group consisting of Bacillus infernus, Defluviitoga tunisiensis, Syntrophaceticus schinkii, Tepidanaerobacter syntrophicus, Tepidimicrobium xylanilyticum and any combination thereof.

In this embodiment, the cryoprotectant comprises glycerol and/or dimethyl sulfoxide. Furthermore, the recovery solution comprises a sufficient amount of nutrient broth. The nutrient broth can be commercially available nutrient broth, such as tryptic soy broth, lysogeny broth, thioglycolate broth, anaerobic basal broth, Wilkins-Chalgren anaerobe broth or any combination thereof.

In a third embodiment, we provide an inoculum composition, comprising anaerobic condition-surviving bacteria for accelerating anaerobic biodegradation of plastics and/or enzyme, wherein the anaerobic condition-surviving bacteria comprises one selected from the group consisting of Bacillus infernus, Defluviitoga tunisiensis, Syntrophaceticus schinkii, Tepidanaerobacter syntrophicus Tepidimicrobium xylanilyticum and any combination thereof.

In this embodiment, the inoculum composition has one or more biochemical characteristics:
(a) the pH of the inoculum composition is in the range of 7.0 to 8.5;
(b) the concentration of volatile fatty acids is 1 g/kg or less; and/or
(c) the concentration of NH₄⁺—N is 2 g/kg or less.

The plastics comprises one selected from the group consisting of polyethylene (PE), polypropylene (PP), polylactic acid (PLA) and any combination thereof. The plastics can be amorphous, semi-crystalline, crystalline form or any combination thereof. Moreover, the shape of the plastics can be film, molded plastic articles, fibers or any other types.

In this embodiment, the product form of the inoculum composition can be liquid suspension, concentrated slurry, frozen culture, freeze-dried powder or tablets, cryopreserved culture or any combination thereof.

In a fourth embodiment, a method for anaerobic biodegradation of plastics is provided. The method comprises adding the inoculum composition of the third embodiment to a system comprising plastics solely or together with anaerobic microbial habitat as bioaugmentation. Preferred, the inoculum composition grows without the addition of external carbon sources in the anaerobic habitats.

In this embodiment, the anaerobic microbial habitat comprises anaerobic soil, anaerobic sludge, landfill compost, sediment or any combination therefore. The dissolved oxygen level of the anaerobic microbial habitat is less than 1 mg/L or less than 1% by weight of the microbial habitat.

In this embodiment, the proportion range of the anaerobic condition-surviving bacteria is more than 1.7×10⁶ cells per 1 g or more than per 1 mL of the whole biodegradation system. Preferred, the bioaugmentation of whole biodegradation system does not require addition of external carbon substances.

Preferred, the inoculum composition is effective at both mesophilic and thermophilic anaerobic conditions. The range of operating temperature is 20 to 60 degrees Celsius.

In a fifth embodiment, we provided an inoculum composition with more additives, also referred to “inoculum matrix”, similar to the inoculum composition in the third embodiment. The “inoculum matrix” further comprises:
(a) enzyme, for enhancing the biodegradation of plastics; and
(b) conditioning agent, for enhancing the performance of enzyme.

In this embodiment, the enzyme comprises hydrolase, lipase, alcalase, laccase, proteinase K, esterase or any combination thereof. The conditioning agent comprises salts, pH buffering, cofactor, electron donor or any combination thereof.

In this embodiment, the inoculum matrix has one or more biochemical characteristics:
(a) the pH of the inoculum composition is in the range of 7.0 to 8.5;
(b) the concentration of volatile fatty acids is 1 g/kg or less; and/or
(c) the concentration of NH4+-N is 2 g/kg or less.

The plastics comprises one selected from the group consisting of polyethylene (PE), polypropylene (PP), polylactic acid (PLA) and any combination thereof. The plastics can be amorphous, semi-crystalline, crystalline form or any combination thereof. Moreover, the shape of the plastics can be film, molded plastic articles, fibers or any other types.

In this embodiment, the product form of the inoculum matrix can be liquid suspension, concentrated slurry, frozen culture, freeze-dried powder or tablets, cryopreserved culture or any combination thereof.

In a sixth embodiment, a method for anaerobic biodegradation of plastics is provided. The method comprises adding the inoculum composition of the fifth embodiment, also referred to “inoculum matrix”, to a system comprising plastics solely or together with anaerobic microbial habitat as bioaugmentation. Preferred, the inoculum composition grows without the addition of external carbon sources in the anaerobic habitats.

In this embodiment, the anaerobic microbial habitat comprises anaerobic soil, anaerobic sludge, landfill compost, sediment or any combination therefore. The dissolved oxygen level of the anaerobic microbial habitat is less than 1 mg/L or less than 1% by weight of the microbial habitat.

In this embodiment, the proportion range of the anaerobic condition-surviving bacteria is more than 1.7×10⁶ cells per 1 g or more than per 1 mL of the whole biodegradation system. Preferred, the bioaugmentation of whole biodegradation system does not require addition of external carbon substances.

Preferred, the inoculum matrix is effective at both mesophilic and thermophilic anaerobic conditions. The range of operating temperature is 20 to 60 degrees Celsius.

The enzyme unit, or international unit for enzyme (symbol U, sometimes also IU) is a unit of enzyme's catalytic activity. 1 U (μmol/min) is defined as the amount of the enzyme that catalyzes the conversion of one micromole of substrate per minute under the specified conditions of the assay method. In this embodiment, the enzyme activity more than 0.48 U per 1 g or 0.48 U per 1 mL of the whole biodegradation system.

### Example 1—Construction of Inoculum Using Bacteria from Various Sources of Anaerobic Microbial Habitat

Enrichment of Various Anaerobic Microbial Habitat with Plastics

One of the applications of the inoculum is for bioaugmentation. The inoculum is designed to be compatible with anaerobic microbial habitat which has indigenous microbes inside. That means the inoculum is still alive or even thrives after addition to the anaerobic habitat for certain period of times, says months.

Several anaerobic microbial habitats were selected in Table 1 for investigating the potential anaerobic condition-surviving bacteria for carrying out the biodegradation of plastics. These anaerobic microbial habitats were the organic matter wastes after anaerobic digestion process from a paper manufacturer factory, a sewage treatment plant and a food waste recovery facility respectively, so that the sources used for the anaerobic biodegradation described in the section below covered wide spectrum of waste feedings, including papers representing industrial wastes, household wastewater and household food wastes.

TABLE 1
Various anaerobic microbial habitats

| Sample ID | Description |
|---|---|
| Soil 1 | Anaerobic sludge from wastewater treatment facility of |
| | a paper manufacturer factory |
| Soil 2 | Anaerobic sludge from sewage Treatment Plant |
| Soil 4 | Anaerobic compost from a food waste recovery facility |

Plastic of PP, PE and PLA were added into the above anaerobic microbial habitats separately and then kept under a standardized anaerobic condition with reference to ASTM D5511. This step can serve as an enrichment step for the anaerobic microbial habitat. The microbial composition will change over time. The bacteria that flavor the condition will thrive. By comparing the change in abundance of microbial species with time, it was aimed to select the microbial species as the candidate potentially accelerating the anaerobic biodegradation of the specific polymers. With this enrichment and selection methodology, microbial consortium found are the species which are able to accelerate the anaerobic biodegradation of plastics, as well as compatible to the indigenous microbes in the anaerobic microbial habitat.

Metagenomic Analysis (16S rRNA and ITS Amplicon Sequencing)

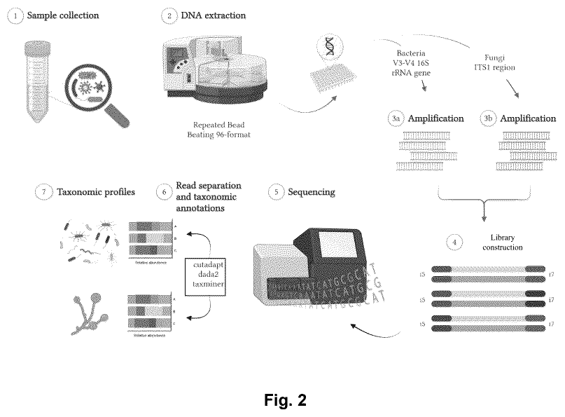

The microbial composition is determined according to the following procedure as described in FIG. 2. The samples (before and after enrichment) collected from the anaerobic microbial habitat were submitted to BGI Tech Solutions (Hong Kong) Co. Limited for 16S rRNA and ITS amplicon sequencing to analyze the possible changes in microenvironment during biodegradation, such as metagenomics, and to identify the species. In brief, the total genomic DNA of the mixed microbes was extracted and purified. 16S rRNA V3-V4 regions and ITS1 region were amplified after polymerase chain reaction (PCR) with target region specific primers. All PCR products are purified by Agencourt AMPure XP beads, dissolved in Elution Buffer and eventually labeled to finish library construction. Library size and concentration were detected by Agilent 2000 Bioanalyzer. Unfortunately, it was failed to construct the ITS libraries due to the harsh environment for fungi growth. Qualified libraries were sequenced on the DNA nanoball (DNB) platform according to their insert size. After clustered to Operational Taxonomic Units (OTU), taxonomic classifications are assigned to OTU representative sequence using Greengene V201306 and Ribosomal Database Project database 11.5 2016 Sep. 30. Data analysis was carried out based on OTU profile table and taxonomic annotation results.

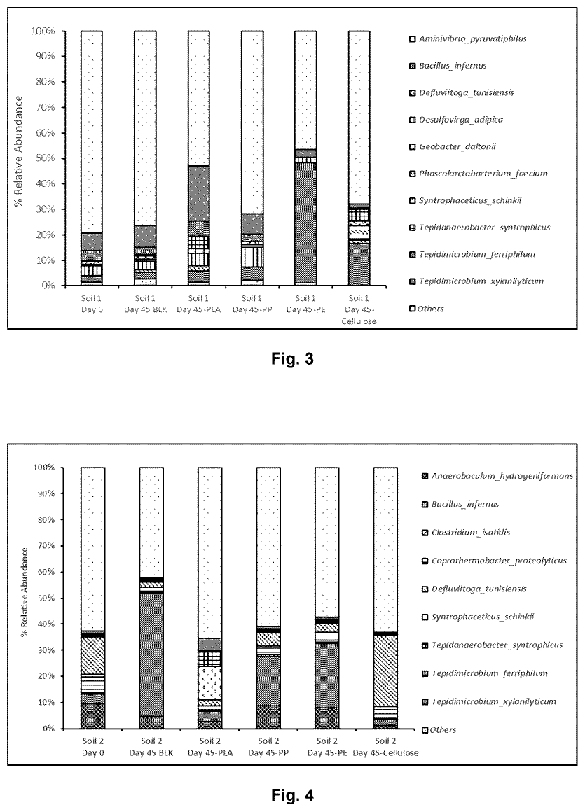

To look for suitable microbes for biodegradation, comparisons of OTU abundance between Day 0 and Day 45 from Soil 1, 2, and 4 with or without PLA, PP, or PE addition were studied separately. The dominant bacteria species in Soil 1, Soil 2, and Soil 4 were reported in FIG. 3 to FIG. 5. In FIG. 3 to FIG. 5, it is noted that cellulose-enrichment is the positive control for testing the soil activity. The results determined are based on the 16S rRNA amplicon sequencing. At the species level, bacteria with relative abundance above 1% were displayed. “Others” in the figures indicates the bacteria species with the sum of relative abundance below 1% or the unidentified species.

Table 2 exhibits increased relative abundance of bacterial community of Soil 1 at day 45 compared to day 0. At the species level, the relative abundance of Bacillus infernus increased dramatically after PE/PP/PLA enrichment at 52 degrees Celsius for 45 days ranging from 81%-1871% in Soil 1 (referring to FIG. 3 and Table 2). Other bacteria, except for Aminivibrio pyruvatiphilus, showed different increases at Day 45 in either PP enrichment, PLA enrichment or both. Moreover, PLA enrichment increased the relative abundance of Defluviitoga tunisiensis, Syntrophaceticus schinkii, and Tepidanaerobacter syntrophicus by over 3000%.

Table 3 exhibits increased relative abundance of bacterial community of Soil 2 at day 45 compared to day 0. In Soil 2, the relative abundance of Tepidimicrobium xylanilyticum elevated in PE/PP/PLA enrichment, while decreased in blank groups at Day 45 (referring to FIG. 4 and Table 3). PLA and PE enrichment increased the relative abundance of Syntrophaceticus schinkii. Especially in the PLA enrichment, the increase reached 5692%. Furthermore, Tepidanaerobacter syntrophicus had a 644% increase in PLA enrichment, meanwhile the reduction of relative abundance of this species in other enrichment and blank groups ranged from −6% to −90%.

Table 4 exhibits increased relative abundance of bacterial community of Soil 4 at day 45 compared to day 0. In Soil 4, the enrichment of all three polymers PE/PP/PLA resulted in an increase of 5847%, 10541%, and 10572% in the abundance of Clostridium alkalicellulosi, which was way higher than the increase in the blank group. Further, various increases of relative abundance were shown in Bacillus infernus, Defluviitoga tunisiensis, Tepidanaerobacter syntrophicus after 45 days of enrichment of PP or PE or PLA (referring to FIG. 5 and Table 4).

In brief, there are 5 bacteria that exhibited increased relative abundance of PE or PP or PLA enrichment in at least two soil samples which are summarized in Table 5.

### Example 2—Cultivation of Inoculum

### On Agar Plate

The species to be potentially included in inoculum is identified in the above metagenomic analysis were further studied for the ability to be cultivated in commercially available nutrient media. 7 commercially available media for anaerobic growth including Columbia blood agar (CBA), tryptic soy agar (TSA), thioglycolate agar (TGA), anaerobic basel agar (ABA), Wilkins-Chalgren anaerobic agar (WCA) and Mannitol egg yolk agar (MYP), Haibo anaerobe agar (HB-ABA) were used for the cultivation of the identified species (Table 6). It was observed that the growth is different for different commercial media. There was no observed growth within 1 month using particular commercial medium.

### In Medium Broth

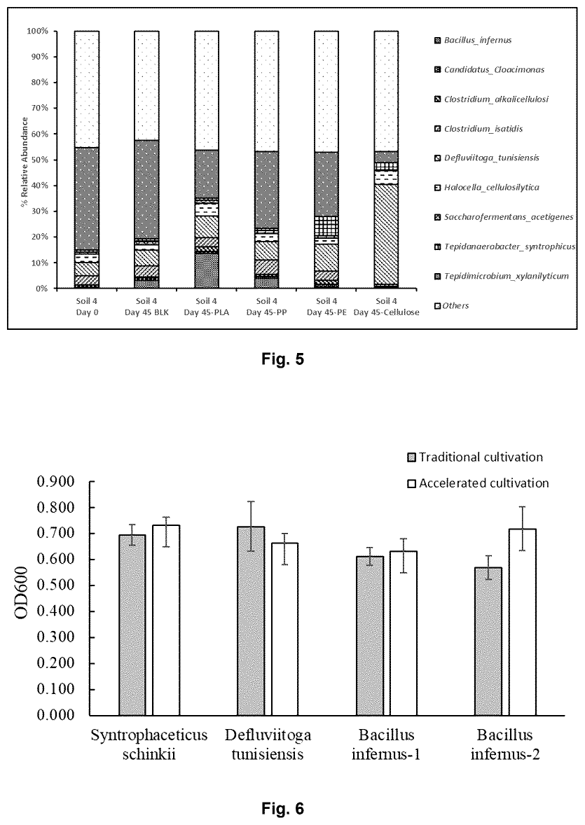

A new culture method is applied in cultivating anaerobic bacteria in this study. 2 mL of anaerobic bacteria glycerol stock (which composition included 75% grown bacteria in nutrient broth and 25% glycerol) is transferred into 100 mL tryptic soy broth (TSB) or other commercial nutrient broth as inoculum. The fresh culture can be used after 24-hour incubation with shaking speed at 220 rpm. Compared to the traditional cultivation method shown in Table 7, the new method can accelerate the culture time by two days. As shown in FIG. 6, the average value of OD600 using accelerated method was very similar to that of using traditional cultivation method. In short, the accelerated cultivation method can shorten the cultivation time of anaerobic bacteria in only two days without affecting the bacterial activity and even can increase the growth.

TABLE 7
The comparison of process and time consuming between
traditional and accelerated cultivation method for anaerobic bacteria.

| Culture method | Day 1 | Day 2 | Day 3 | Day 4 |
|---|---|---|---|---|
| Conventional | Streak bacteria glycerol | Inoculate one loop | Transfer 3 mL | 100 mL |
| cultivation | stock (stored at −80° C.) | of bacteria lawn | bacteria culture into | bacteria |
| (Stepwise) | on an agar plate by using | from the agar plate | 100 mL TSB after 24 | culture is ready |
| | inoculation loop and | into 3 mL of TSB. | hours of cultivation. | for use. |
| | cultivate. Incubate in an | Shake at 220 rpm in | Shake at 220 rpm in | |
| | anaerobic growth | an anaerobic | an anaerobic growth | |
| | chamber at 30° C. for 24 | growth chamber at | chamber at 30° C. for | |
| | hours. | 30° C. for 24 hours. | 24 hours. | |
| Accelerated | Transfer 4 mL of bacteria | 100 mL bacteria | | |
| cultivation | glycerol stock (stored at | culture is ready for | | |
| | −80° C.) into 100 mL TSB. | use. | | |
| | Shake at 220 rpm in an | | | |
| | anaerobic growth | | | |
| | chamber at 30° C. for 24 | | | |
| | hours. | | | |

### Example 3—Application of Inoculum for Acceleration for biodegradation

Series of anaerobic biodegradation experiments with each of PP, PE and PLA as test specimens in anaerobic soil were carried out in order to investigate the acceleration in biodegradation of plastics by the inoculum with reference to a standardized anaerobic testing method for plastics (ASTM D5511).

### Preconditioning Procedures of the Anaerobic Soil Fulfilling the Requirement of ASTM D-5511

Various anaerobic soils were collected from different sources of origin. However, some were excluded from the study as their physical properties were different from the typical anaerobic soil, such as no biogas activity. So far, 3 inoculum soil sources (namely, Soil 1, Soil 2, and Soil 4) were selected for the series of experiments. These anaerobic soil sources were the organic matter wastes after anaerobic digestion process from a paper manufacturer factory, a sewage treatment plant and a food waste recovery facility respectively, so that the inoculum soil sources used for the anaerobic biodegradation described in the section below covered wide spectrum of waste feedings, including papers representing industrial wastes, household wastewater and household food wastes. With using anaerobic soil sources from different anaerobic digestion plants, it was not only aimed to investigate the inoculum composition for accelerating the anaerobic biodegradation of plastics, but also the compatibility of the inoculum composition when it is added in the actual soil sources. The details are listed in Table 8. Upon arrival, inoculum soil was concentrated by centrifugation to reach a dry solid content of at least 20% if the water content was too high, followed by storage under thermophilic conditions (52±2 degrees Celsius) for at least 7 days without feeding before starting the anaerobic biodegradation to reduce the background biodegradation of the inoculum soil as described in ASTM D5511. Dry solid content and pH value before anaerobic biodegradation experiments were measured.

TABLE 8

| Sample | | Dry Solid Content | pH |
|---|---|---|---|
| ID | Description | (%) | Value |
| Soil 1 | Anaerobic sludge from | 20.8% | 8.20 |
| | wastewater treatment facility of | | |
| | a paper manufacturer factory | | |
| Soil 2 | Anaerobic sludge from sewage | 21.2% | 8.33 |
| | Treatment Plant | | |
| Soil 4 | Anaerobic compost from a food | 20.1% | 8.21 |
| | waste recovery facility | | |

### Carbon Content of Test Specimens

Biodegradation refers to the percentage conversion of organic carbon (in terms of total organic carbon (TOC)) of a test specimen to gaseous carbon (CH₄ and CO₂) by means of microbial degradation. TOC content of the test specimens, including commercially available PP, PE, and PLA pellets and thin-layer chromatography (TLC) cellulose (as positive control), was measured by elemental analyzer and summarized in Table 9.

TABLE 9

| | Total Organic Carbon (TOC) |
|---|---|
| Test Specimens | Content (%) |
| PP | 78.95% |
| PE | 78.76% |
| PLA | 45.53% |
| Thin-layer chromatography (TLC) | 38.60% |
| cellulose | |

### General Procedure for Biodegradation Test

Each setup for anaerobic biodegradation consists of a bioreactor and a biogas collector which was assembled based on the description in ASTM D5511 with minor amendment in setup as following. Glass cultivation bottle of size of 600 mL equipped with 4-port connection screw cap was used as the bioreactor, which was connected to a biogas collector though the opening port on the screw cap as shown in FIG. 7A and FIG. 7B. Biogas released from the reactor was thus collected by an inverted cylinder in acid bath (diluted HCl, pH <2) and its volume was measured by water displacement method.

5 experimental setups, including (1) blank (anaerobic soil only for measuring the background biodegradation); (2) positive control (TLC cellulose); (3) PP; (4) PE; and (5) PLA (NatureWorks 3001D), were carried out for each anaerobic soil (Soil 1, Soil 2 and Soil 4). In general, 300 g of the above anaerobic soil and 30 g test specimen were added to the bioreactor and mixed carefully so that the test specimen was evenly distributed in the inoculum soil. Nitrogen gas was purged for at least 5 minutes to remove oxygen, followed by immediate connection to the biogas collector. Gas leakage checking was required to ensure anaerobic environment and biogas collection. The experiment was carried out under thermophilic conditions (52±2 degrees Celsius).

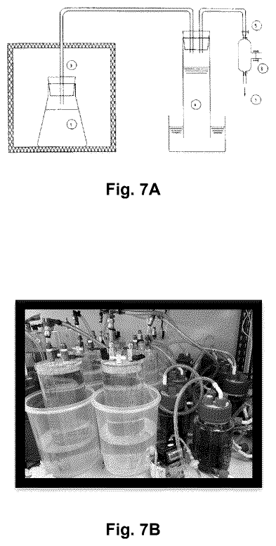

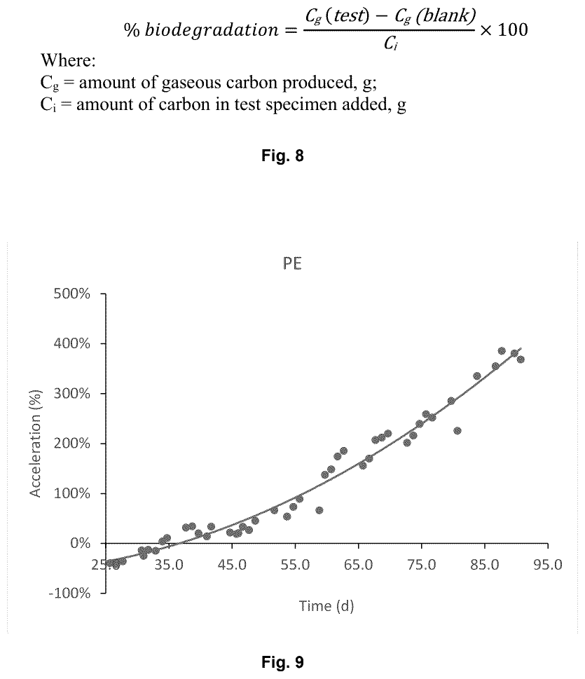

The total volume of the collected biogas was recorded. Concentration of methane (CH₄) and carbon dioxide (CO₂) in the biogas were measurement by a biogas analyzer. Room temperature and atmospheric pressure were recorded in every biogas volume recording for the normalization of the measured volume at ambient condition to standard temperature and pressure (STP). Determination of biodegradability is described in FIG. 8.

### Accelerated Anaerobic Biodegradation of PE

An inoculum composition of microbial consortium containing 3 types of bacterial Bacillus infernus (no. of cell: 5×10⁹), Defluviitoga tunisiensis (no. of cell: 0) and Syntrophaceticus schinkii (no. of cell: 5×10⁸) were cultivated in phosphate buffer (6 mL) to form a microbial consortium solution. 2 experimental setups, including (1) blank (300 g inoculum soil plus the microbial consortium solution for measuring the background biodegradation); (2) 30 g PE plus the microbial consortium solution were carried out under thermophilic conditions (52° C.±2 degrees Celsius) for at least 90 days. Triplicates of each setup were performed. The acceleration of the anaerobic biodegradation was monitored during the experiment period by dividing the percentage biodegradation of PE in presence of the inoculum composition solution by the percentage biodegradation of the control (no inoculum composition added). The biodegradation rate of PE at day 90 was accelerated by 368% in presence of the microbial consortium solution (FIG. 9).

### Accelerated Anaerobic Biodegradation of PP

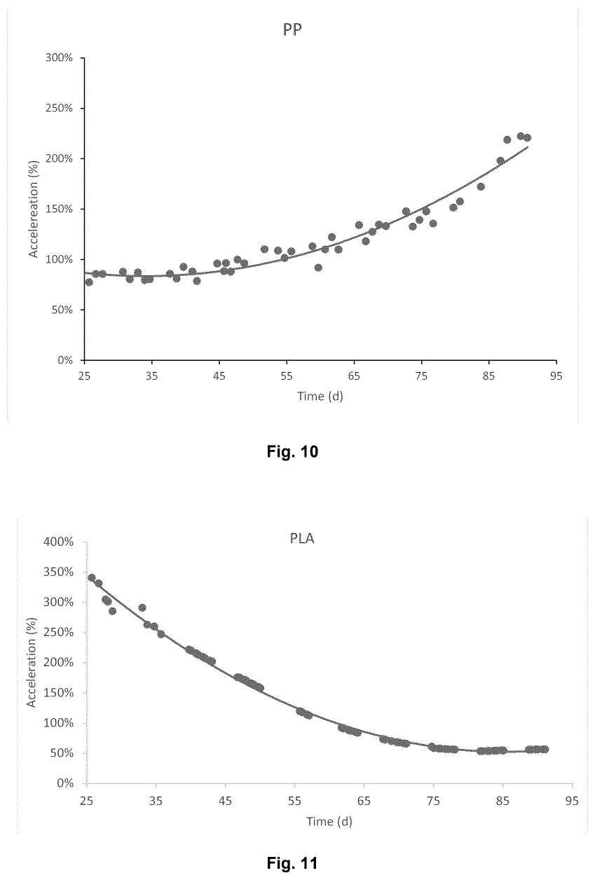

An inoculum composition of microbial consortium containing 3 types of bacterial, Bacillus infernus (no. of cell: 5×10⁹), Defluviitoga tunisiensis (no. of cell: 0) and Syntrophaceticus schinkii (no. of cell: 5×10⁹) were cultivated in phosphate buffer (6 mL) to form a microbial consortium solution. 2 experimental setups, including (1) blank (300 g inoculum soil plus the microbial consortium solution for measuring the background biodegradation); (2) 30 g PP plus the microbial consortium solution were carried out under thermophilic conditions (52° C.±2 degrees Celsius) for at least 90 days. Triplicates of each setup were performed. The acceleration of the anaerobic biodegradation was monitored during the experiment period by dividing the percentage biodegradation of PP in presence of the microbial consortium solution by the percentage biodegradation of the control (no microbial consortium added). The biodegradation rate of PP was accelerated by 234% in presence of the microbial consortium solution (FIG. 10).

### Accelerated Anaerobic Biodegradation of PLA

An inoculum composition of microbial consortium containing 3 types of bacterial, Bacillus infernus (no. of cell: 0), Defluviitoga tunisiensis (no. of cell: 5×10⁸) and Syntrophaceticus schinkii (no. of cell: 5×10⁹) were cultivated in phosphate buffer (6 mL) to form a microbial consortium solution. 2 experimental setups, including (1) blank (300 g inoculum soil plus the microbial consortium solution for measuring the background biodegradation); (2) 30 g PLA (NatureWorks 4060D, pellets) plus the microbial consortium solution were carried out under thermophilic conditions (52±2 degrees Celsius) for at least 90 days. Triplicates of each setup were performed. The acceleration of the anaerobic biodegradation was monitored during the experiment period by dividing the percentage biodegradation of PLA in presence of the microbial consortium solution by the percentage biodegradation of the control (no microbial consortium added). The biodegradation rate of PLA was accelerated by 56% in presence of the microbial consortium solution (FIG. 11)

### Example 4—Application of Enzyme for Acceleration of Biodegradation

Control Experiment for Comparison: (without Enzyme as Control)

2 experimental setups, including (1) blank (inoculum soil only for measuring the background biodegradation); (2) PLA films (NatureWorks 4060 D, size: 2×2 cm, thickness 100-150 μm) were carried out. Triplicates of each setups were performed. In general, 300 g of the above inoculum soil and 30 g PLA films were added to the bioreactor and mixed carefully so that the test specimen was evenly distributed in the inoculum soil. Nitrogen gas was purged for at least 5 minutes to remove oxygen, followed by immediate connection to the biogas collector. Gas leakage checking was required to ensure anaerobic environment and biogas collection. The experiment was carried out under thermophilic conditions (52±2 degrees Celsius) for at least 90 days. The total volume of the collected biogas was recorded. Concentration of methane (CH₄) and carbon dioxide (CO₂) in the biogas were measured. Room temperature and atmospheric pressure were recorded in every biogas volume recording for the normalization of the measured volume at ambient condition to standard temperature and pressure (STP).

### Enzyme 1-Lipase

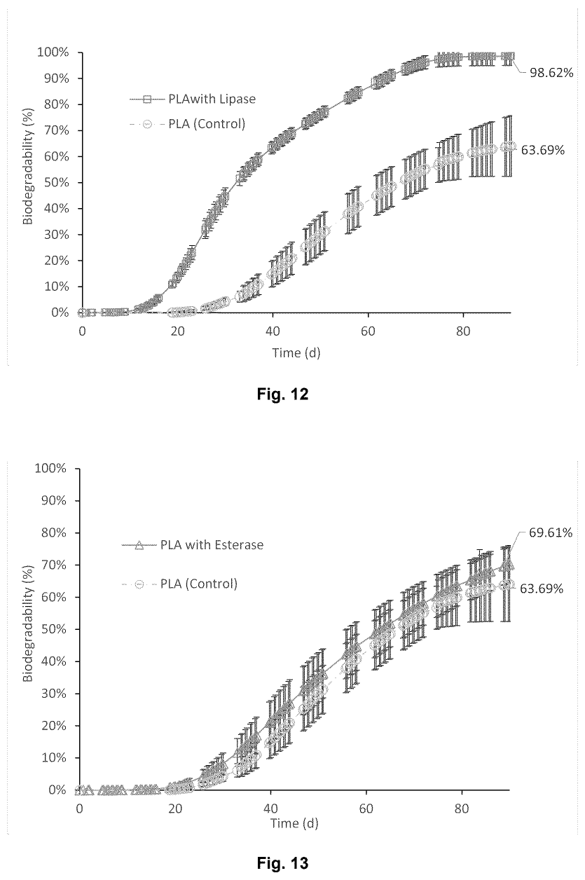

A solution of lipase with activity of 6000 U in pH 7 phosphate buffer (6 mL) was prepared for each setup. 2 experimental setups, including (1) blank (300 g inoculum soil plus the said lipase solution for measuring the background biodegradation); (2) 30 g PLA films soil plus the said solution of lipase were carried out under thermophilic conditions (52±2 degrees Celsius) for at least 90 days. Triplicates of each setup were performed. The acceleration of the anaerobic biodegradation was monitored during the experiment period by dividing the percentage biodegradation of PLA in presence of lipase solution by the percentage biodegradation of the control (no enzyme added). Anaerobic biodegradation was accelerated by 54.6% in 90 days in the presence of lipase (FIG. 12).

### Enzyme 2-Esterase

A solution of esterase with activity of 500 U in pH 7 phosphate buffer (5 mL) was prepared for each setup. 2 experimental setups, including (1) blank (300 g inoculum soil plus the said esterase solution for measuring the background biodegradation); (2) 30 g PLA films soil plus the said solution of esterase were carried out under thermophilic conditions (52±2 degrees Celsius) for at least 90 days. Triplicates of each setup were performed. The acceleration of the anaerobic biodegradation was monitored during the experiment period by dividing the percentage biodegradation of PLA in presence of esterase solution by the percentage biodegradation of the control (no enzyme added). Anaerobic biodegradation was accelerated by 9.4% in 90 days in the presence of esterase (FIG. 13).

### Enzyme 3-Alcalase

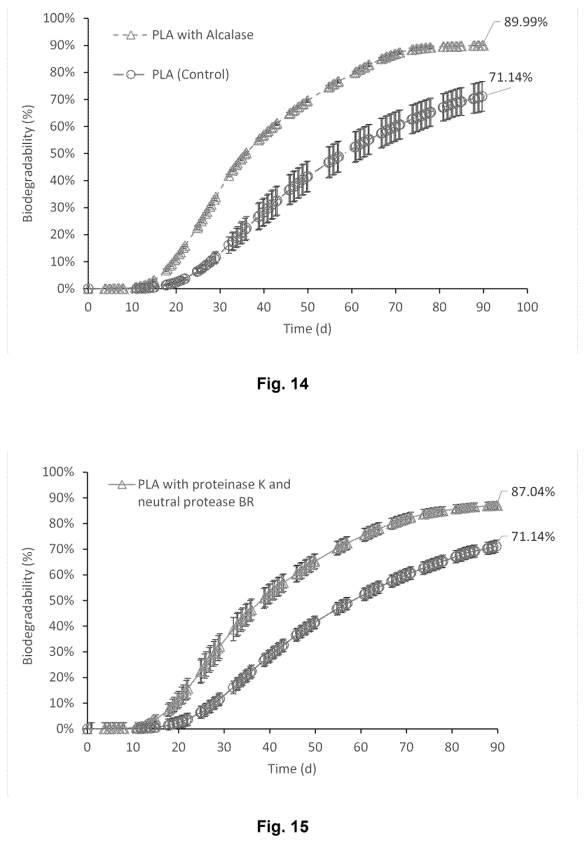

A solution of alcalase with activity of 15 AU/g in pH 7 phosphate buffer (5.644 mL) was prepared for each setup. 2 experimental setups, including (1) blank (300 g inoculum soil plus the said alcalase solution for measuring the background biodegradation); (2) 30 g PLA films soil plus the said solution of alcalase were carried out under thermophilic conditions (52±2 degrees Celsius) for at least 90 days. Triplicates of each setup were performed. The acceleration of the anaerobic biodegradation was monitored during the experiment period by dividing the percentage biodegradation of PLA in presence of alcalase solution by the percentage biodegradation of the control (no enzyme added). Anaerobic biodegradation was accelerated by 26.5% in 90 days in the presence of alcalase (FIG. 14)

### Enzyme 4-Proteinase K and Neutral Protease BR

A solution of proteinase K and neutral protease BR with activity of 100 U and 6000 U respectively in pH 7 phosphate buffer (0.1 mL and 3 ml respectively) was prepared for each setup. 2 experimental setups, including (1) blank (300 g inoculum soil plus the said proteinase K and neutral protease BR solution for measuring the background biodegradation); (2) 30 g PLA films soil plus the said solution of said proteinase K and neutral protease BR were carried out under thermophilic conditions (52±2 degrees Celsius) for at least 90 days. Triplicates of each setup were performed. The acceleration of the anaerobic biodegradation was monitored during the experiment period by dividing the percentage biodegradation of PLA in presence of proteinase K and neutral protease BR solution by the percentage biodegradation of the control (no enzyme added). Anaerobic biodegradation was accelerated by 22.4% in 90 days in the presence of proteinase K and neutral protease BR (FIG. 15).

### Example 5—Application of Inoculum Matrix for Acceleration of Biodegradation

An inoculum composition of microbial consortium solution was prepared by cultivation of 3 types of bacteria, Bacillus infernus (no. of cell: 0), Defluviitoga tunisiensis (no. of cell: 5×10⁸) and Syntrophaceticus schinkii (no. of cell: 5×10⁹) in phosphate buffer (6 mL). An enzyme solution was prepared with lipase with activity of 6000 U in phosphate buffer (6 mL). 4 experimental setups, including (1) blank (inoculum soil only for measuring the background biodegradation); (2) PLA (NatureWorks 4060D, pellets) (control); (3) PLA with microbial consortium solution (also referred to “inoculum composition”); and (4) PLA with microbial consortium solution and enzyme solution (also referred to “inoculum matrix”) were carried out. Triplicates of each setup were performed. In general, 300 g of the above inoculum soil and 30 g PLA were added to the bioreactor and mixed carefully so that the test specimen was evenly distributed in the inoculum soil. The microbial consortium solution and the enzyme solution were added to the corresponding setup. Nitrogen gas was purged for at least 5 minutes to remove oxygen, followed by immediate connection to the biogas collector. Gas leakage checking was required to ensure anaerobic environment and biogas collection. The experiment was carried out under thermophilic conditions (52±2 degrees Celsius) for at least 90 days. The total volume of the collected biogas was recorded. Concentration of methane (CH₄) and carbon dioxide (CO₂) in the biogas were measured. Room temperature and atmospheric pressure were recorded in every biogas volume recording for the normalization of the measured volume at ambient condition to standard temperature and pressure (STP). Percentage of biodegradation of PLA in presence of microbial consortium solution was 81.4%, while PLA without microbial consortium solution was 52.4%. Biodegradation of PLA was increased to 83.1% in presence of both microbial consortium solution and enzyme solution. Biodegradation rate of PLA was accelerated by 55.3% in presence of microbial consortium solution and 58.6% in presence of microbial consortium solution and enzyme solution respectively (FIG. 16).

### Example 6—Biodegradation of PLA Using Different Combinations of Bacteria in Inoculum Composition

### General Experimental Setup

Each setup for anaerobic biodegradation consists of a bioreactor and a biogas collector which was assembled based on the description in ASTM D5511 with minor amendment in setup as follows. Glass cultivation bottle of size of 600 mL equipped with 4-port connection screw cap was used as the bioreactor, which was connected to a biogas collector though the opening port on the screw cap. Biogas released from the reactor was thus collected by an inverted cylinder in acid bath (diluted HCl, pH<2) and its volume was measured by water displacement method.

### Accelerated Anaerobic Biodegradation of Plastics in Presence of Inoculum Composition

At least 6 combinations of inoculum compositions were used for the optimization experiment. For each combination, the inoculum composition was cultivated in phosphate buffer (6 mL) to form a microbial consortium solution. 2 experimental setups, including (1) blank (100 g inoculum soil plus the microbial consortium solution for measuring the background biodegradation); (2) 10 g plastic specimen plus the microbial consortium solution were carried out under thermophilic conditions (52±2 degrees Celsius) for at least 30 days. Triplicates of each setup were performed. The percentage biodegradation of the plastic specimens was compared with the corresponding control experiment to obtain the rate of acceleration of the anaerobic biodegradation of the plastic specimen.

### Optimization of Inoculum Compositions for PLA

The optimization of inoculum composition was performed by statistically based experimental designs. Series of anaerobic biodegradation experiments were carried out according to the general procedure described above with addition of different combinations of microbial consortium as the fractional factorial design experiments. The possible combinations set for the inoculum composition were listed in Table 10. The final biodegradability results of plastics of this series of experiments were regressed by statistical software such as Minitab to analyze the significant effects on the anaerobic biodegradation of plastics.

The optimization of inoculum compositions for PLA (NatureWorks 4060D, pellets) was performed for 45 days. The combination of microbial consortium, and the rate of biodegradation of PLA in each of the combinations at day 45 were listed in Table 9. Different rate of biodegradation of PLA has been resulted and the combination no. 8, containing Syntrophaceticus schinkii at cell count of 1.7×10⁹ level, Defluviitoga tunisiensis at cell count of 1.7×10⁸ level and Bacillus infernus at cell count of 1.7×10⁹, gave the highest percentage biodegradation of PLA in 45 days (Table 10).

TABLE 10
Formulations of inoculum compositions in 100 g of inoculum

| | Syntrophaceticus | Defluviitoga | Bacillus | Day 45 Biodegradability |
|---|---|---|---|---|
| | schinkii | tunisiensis | infernus | (%) |
| 1 | 0 | 0 | 0 | 41.0 ± 14.0 |
| 2 | 0 | 1.7 × 10⁸ | 1.7 × 10⁸ | 49.0 ± 0.8 |
| 3 | 0 | 1.7 × 10⁹ | 1.7 × 10⁹ | 42.9 ± 4.0 |
| 4 | 1.7 × 10⁸ | 0 | 1.7 × 10⁸ | 37.5 ± 8.2 |
| 5 | 1.7 × 10⁸ | 1.7 × 10⁸ | 1.7 × 10⁹ | 40.1 ± 11.8 |
| 6 | 1.7 × 10⁸ | 1.7 × 10⁹ | 1.7 × 10⁸ | 35.1 ± 1.4 |
| 7 | 1.7 × 10⁹ | 0 | 1.7 × 10⁹ | 43.1 ± 2.5 |
| 8 | 1.7 × 10⁹ | 1.7 × 10⁸ | 1.7 × 10⁸ | 51.4 ± 2.0 |
| 9 | 1.7 × 10⁹ | 1.7 × 10⁹ | 1.7 × 10⁹ | 48.9 ± 0.9 |

Results were subjected to statistical analysis by DOE method using softwares Minitab to look for the microbial species or combination which have significant effect on anaerobic biodegradation of PLA, and the results obtained from Minitab showed that the presence of species Syntrophaceticus schinkii at cell count of 1.7×10⁹ level and the species Defluviitoga tunisiensis at cell count of 1.7×10⁸ level had the most significant effect, while the biodegradation of PLA were very similar with or without additions of Bacillus infernus. Therefore, in summary the optimized inoculum composition resulted in this experiment was shown in Table 11.

TABLE 11
The optimized inoculum composition for anaerobic
biodegradation of PLA in 100 g inoculum soil

| Microbial species | No. of cell count (in 100 g inoculum soil) |
|---|---|
| Syntrophaceticus schinkii | 1.7 × 10⁹ |
| Defluviitoga tunisiensis | 1.7 × 10⁸ |
| Bacillus infernus | 0 |

### Example 7—Optimization of Enzymes in Inoculum Matrix

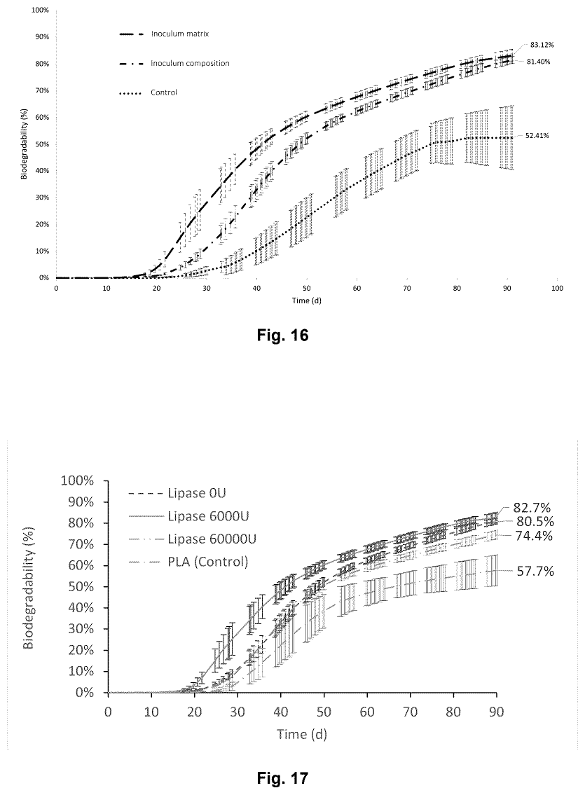

The optimized level of enzyme activity was investigated by adding 3 different levels of lipase together with one inoculum composition (which also referred to “inoculum matrix”) into an inoculum soil for anaerobic biodegradation of PLA. 3 levels of lipase solution of lipase with enzyme activity of (0 U, 6000 U and 60000 U) in phosphate buffer (6 mL) was prepared respectively. For each level of lipase, 2 experimental setups, including (1) blank (300 g inoculum soil plus the said lipase solution for measuring the background biodegradation); (2) 30 g PLA (NatureWorks 4060D, pellets), inoculum soil plus the said solution of lipase were carried out under thermophilic conditions (52±2 degrees Celsius) for at least 90 days. Triplicates of each setup were performed. For comparison with genuine biodegradation rate of PLA in the same inoculum soil, a pair of control experiment setups including (1) blank (300 g inoculum soil); (2) 30 g PLA (NatureWorks 4060D, pellets) were also carried with triplicates. After 90-day experiment period, the highest biodegradation rate of was obtained in presence of 6000 U lipase (FIG. 17). Therefore, 6000 U of lipase was selected as the optimized enzyme activity for lipase for the anaerobic biodegradation of PLA.

### Example 8—Stability of the Inoculum Composition for acceleration of Biodegradation

The inoculum soil with additional inoculum composition has been sent for 16S rRNA amplicon sequencing analysis on day 0 and day 90 of the said accelerated anaerobic biodegradation of PLA in Example 3. The changes of relative abundance of the 3 types of bacteria in the inoculum composition (at species level) after 90-day anaerobic biodegradation of PLA were shown in Table 12. All bacteria species, namely Bacillus infernus, Defluviitoga tunisiensis and Syntrophaceticus schinkii, survived after 90-day biodegradation with increase in in relative abundance from 0.01%, 0.01% and 0.04% to 0.06%, 0.53% and 0.36% respectively. This shows that the spiked bacteria not only survive in the anaerobic soil with the indigenous microbes but also thrive in the soil.

TABLE 12
Relative abundance of the 3 types of bacteria species on day
0 and day 90 of the anaerobic biodegradation of PLA
in presence of inoculum compositions of Example 7

| | Relative Abundance | |
|---|---|---|
| | Day 0 | Day 90 |
| Bacillus infernus | 0.01% | 0.06% |
| Defluviitoga tunisiensis | 0.01% | 0.53% |
| Syntrophaceticus schinkii | 0.04% | 0.36% |

The above embodiments are only used to illustrate the principles of the present invention, and they should not be construed as limiting the present invention in any way. The above embodiments can be modified by those with ordinary skill in the art without departing from the scope of the present invention as defined in the following appended claims.

What is claimed is:

1. An inoculum composition, comprising anaerobic condition-surviving bacteria for accelerating anaerobic biodegradation of plastics and/or enzyme, wherein the anaerobic condition-surviving bacteria comprises one selected from the group consisting of Bacillus infernus, Defluviitoga tunisiensis, Syntrophaceticus schinkii, Tepidanaerobacter syntrophicus Tepidimicrobium xylanilyticum and any combination thereof.

2. The inoculum composition of claim 1, wherein the inoculum composition has one or more biochemical characteristics:
(a) the pH of the inoculum composition is in the range of 7.0 to 8.5;
(b) the concentration of volatile fatty acids is 1 g/kg or less; and/or
(c) the concentration of NH₄⁺—N is 2 g/kg or less.

3. The inoculum composition of claim 1, wherein the plastics comprises one selected from the group consisting of polyethylene (PE), polypropylene (PP), polylactic acid (PLA) and any combination thereof.

4. The inoculum composition of claim 1, wherein the product form of the inoculum composition comprises liquid suspension, concentrated slurry, frozen culture, freeze-dried powder or tablets, cryopreserved culture or any combination thereof.

5. A method for anaerobic biodegradation of plastics, comprising adding the inoculum composition of claim 1 to a system comprising plastics solely or together with anaerobic microbial habitat as bioaugmentation.

6. The method of claim 5, wherein the anaerobic microbial habitat comprises anaerobic soil, anaerobic sludge, landfill compost, sediment or any combination therefore.

7. The method of claim 5, wherein the dissolved oxygen level of the anaerobic microbial habitat is less than 1 mg/L or less than 1% by weight of the microbial habitat.

8. The method of claim 5, wherein the proportion range of the anaerobic condition-surviving bacteria is more than 1.7×10⁶ cells per 1 g or more than per 1 mL of the whole biodegradation system.

9. The method of claim 5, wherein the inoculum composition grows without the addition of external carbon sources in the anaerobic habitats.

10. The method of claim 5, wherein the bioaugmentation of whole biodegradation system does not require addition of external carbon substances.

11. The method of claim 5, wherein the operating temperature of the anaerobic biodegradation of plastics ranges from 20 to 60 degrees Celsius.

12. The inoculum composition of claim 1, wherein the inoculum composition further comprises:
(a) enzyme, for enhancing the biodegradation of plastics; and
(b) conditioning agent, for enhancing the performance of enzyme.

13. The inoculum composition of claim 12, wherein the enzyme comprises hydrolase, lipase, alcalase, laccase, proteinase K, esterase or any combination thereof.

14. The inoculum composition of claim 12, wherein the conditioning agent comprises salts, pH buffering, cofactor, electron donor or any combination thereof.

15. A method for anaerobic biodegradation of plastics, comprising adding the inoculum composition of claim 12 to a system comprising plastics solely or together with anaerobic microbial habitat as bioaugmentation.

16. The method of claim 15, wherein the enzyme activity more than 0.48 U per 1 g or 0.48 U per 1 mL of the whole biodegradation system.

17. A method for increasing the proliferative rate of anaerobic condition-surviving bacteria which have been subjected to cryopreservation with cryoprotectant to form a freezing solution, comprising:
(a) adding a first amount of the freezing solution into a second amount of recovery solution to form a mixture solution, so as to thaw the anaerobic condition-surviving bacteria, wherein the second amount is 10 times or more than the first amount; and
(b) culturing the thawed anaerobic condition-surviving bacteria in the mixture solution;
wherein the mixture solution is substantially free of hydrogel.

18. The method of claim 17, wherein the method is a one-pot process to cultivate the anaerobic condition-surviving bacteria.

19. The method of claim 17, wherein the anaerobic condition-surviving bacteria comprises one selected from the group consisting of Bacillus infernus, Defluviitoga tunisiensis, Syntrophaceticus schinkii, Tepidanaerobacter syntrophicus, Tepidimicrobium xylanilyticum and any combination thereof.

20. The method of claim 17, wherein the cryoprotectant comprises glycerol and/or dimethyl sulfoxide.

21. The method of claim 17, wherein the recovery solution comprises a sufficient amount of nutrient broth.

22. The method of claim 17, wherein the anaerobic condition-surviving bacteria are amplified within 1 week from directly sub-culturing the thawed cryopreserved culture.
